(19) United States
(12) Patent Application Publication
(10) Pub. No.: US 2023/0289619 A1
(43) Pub. Date: Sep. 14, 2023

(54) ADAPTIVE DATA MODELS AND SELECTION THEREOF

(71) Applicant: BenevolentAI Technology Limited, London (GB)

(72) Inventors: Rachel HODOS, Staten Island, NY (US); Yingkai GAO, Brooklyn, NY (US); Daniel Lawrence NEIL, Williamsburg, VA (US); Pierre-Louis Maurice Valentin CEDOZ, New York, NY (US)

(21) Appl. No.: 18/040,538

(22) PCT Filed: Aug. 4, 2021
(86) PCT No.: PCT/GB2021/052013

§ 371 Date: Feb. 3, 2023

Related U.S. Application Data
(60) Provisional application No. 63/061,302, filed on Aug. 5, 2020.

(51) Int. Cl. G06N 5/02

(52) U.S. Cl. CPC G06N 5/02

(57) ABSTRACT

Method(s), apparatus, and system(s) are provided for selecting a data model configuration for use in training predictive models comprise receiving two or more data model configurations, extracting a data model for each of the two or more data model configurations from a knowledge graph, generating a separate predictive model for each of the extracted data models, scoring the output of each separate predictive model based on a benchmark data set, and selecting at least one data model configuration of the two or more data model configurations based on the output scores.

The present application relates to a system, apparatus and method(s) for specifying, evaluating, and selecting a data model configuration for use in training one or more machine learning (ML) predictive models and the like configured for receiving knowledge graph information as input and for providing trained ML predictive model(s) based on said selected data model configuration.

### BACKGROUND

Knowledge graphs are increasingly prevalent tools that can be used to infer new relationships between entities. Data in knowledge graphs can be represented in various ways; typically, nodes can be used to represent entities, and relationships between these entities can be represented as edges. In particular, they can be employed in the field of drug development to infer hitherto unknown relationships between, without limitation, for example genes and diseases. This is often performed by trained machine learning (ML) models that accept a knowledge graph as input, and can output newly inferred relationships.

In practice, the prediction of new inferences is often performed on subsets of large knowledge graphs in order to reduce so-called noise and the inference of false-positive relationships where none exist. Prior to inferring relationships based on an input knowledge graph or subset thereto, an ML predictive model may be trained on similar subsets of the knowledge graph and subsequently, once trained, applied to as hitherto unseen subsets of the knowledge graph for inferring new relationships and the like therefrom. The creation of the subsets of the knowledge graph or extraction of a subset from the knowledge graph (also known and referred to herein, as a ‘data model’) can be performed according to any number of conventional methods.

Each data model may comprise or represent data representative of a subset of the knowledge graph and may be extracted from the knowledge graph based on a data model configuration. The data model configuration may comprise or represent data representative of one or more conditions, parameters, values, criteria, relationships, entities, confidence scores, or any other data, node, edge or attribute representing the knowledge graph that may be used for defining and extracting the subset knowledge graph from the knowledge graph. For instance, the edges in the knowledge graph may have associated attributes that, for example, indicate confidence scores for the relationship. In this case, a decision process can be used to define a data model configuration that is used to decide the proportion of edges used to generate a data model for use in inferring new relationships; i.e. a percentage of highest confidence scores is selected while the rest of the full knowledge graph is excluded. Another example may be defining a data model configuration based on a selection of a limited number of types of relationship; for example, in a biomedical domain, the data model may consist only of the subset of the total knowledge graph where entities are related by an edge indicating that a gene ‘causes’ a disease. Currently choosing or defining appropriate data model configuration(s) for filtering, extracting, or deciding which portions or a subset of the knowledge graph are to be used is a manual, ad hoc process that is extremely time-consuming and error-prone.

There is a desire for a more efficient and robust system for generating and selecting a data model from a knowledge graph for optimising the training of one or more ML predictive model(s) that result in the downstream workflow in robust ML predictive model(s) for inferring relationships and the like from an ever-changing and/or updated knowledge graph and the like. There is a further desire for such a system to enable rapid experimentation, optimisation, and selection of different data model configurations for ensuring the best data model configuration, and hence the best data model, is appropriately chosen for improving the predictive accuracy of downstream ML predictive model(s) trained on and/or applied to such selected data model(s) and improved accuracy of predictions output therefrom (e.g. genes for a query disease).

The embodiments described below are not limited to implementations which solve any or all of the disadvantages of the known approaches described above.

### SUMMARY

This Summary is provided to introduce a selection of concepts in a simplified form that are further described below in the Detailed Description. This Summary is not intended to identify key features or essential features of the claimed subject matter, nor is it intended to be used to determine the scope of the claimed subject matter; variants and alternative features which facilitate the working of the invention and/or serve to achieve a substantially similar technical effect should be considered as falling into the scope of the invention disclosed herein.

The present disclosure describes a system for specifying, testing, evaluating, and selecting data models based on the predictive performance (or other properties) of corresponding predictive ML models that are trained using the information specified by each of the data models. This system can greatly streamline a process that would otherwise be inefficient and especially in scenarios where it is unclear which parts or subsets of a knowledge graph would be optimally suited to support a given ML task, such as prediction of links between genes and diseases. In turn, the overall predictive performance shall be significantly improved such that more accurate predictive ML models can be derived from the selected data models or data model configurations.

In a first aspect, the present disclosure provides a computer-implemented method of selecting a data model configuration for use in training predictive models comprising: receiving two or more data model configurations; extracting a data model for each of the two or more data model configurations from a knowledge graph; generating a separate predictive model for each of the extracted data models; scoring the output of each separate predictive model based on a benchmark data set; and selecting at least one data model configuration of the two or more data model configurations based on the output scores.

In a second aspect, the present disclosure provides a computer-implemented method for training a separate predictive model for each of two or more data model configurations comprising: extracting a set of training data for each of the two or more data model configuration from a knowledge graph; and training the separate predictive model using the set of training data.

In a third aspect, the present disclosure provides an apparatus for selecting a data model configuration, the apparatus comprising: an input component configured to receive two or more data model configurations; a processing component configured to extract a data model for each of the two or more data model configurations from a knowledge graph; a prediction component configured to generate a separate predictive model for each of the data models; a scoring component configured to score output from each of the separate predictive model based on a benchmark data set; and a selection component configured to select the data model configuration of the two or more data model configurations based on the scoring

The methods described herein may be performed by software in machine-readable form on a tangible storage medium e.g. in the form of a computer program comprising computer program code means adapted to perform all the steps of any of the methods described herein when the program is run on a computer and where the computer program may be embodied on a computer-readable medium. Examples of tangible (or non-transitory) storage media include disks, thumb drives, memory cards etc. and do not include propagated signals. The software can be suitable for execution on a parallel processor or a serial processor such that the method steps may be carried out in any suitable order, or simultaneously.

This application acknowledges that firmware and software can be valuable, separately tradable commodities. It is intended to encompass software, which runs on or controls “dumb” or standard hardware, to carry out the desired functions. It is also intended to encompass software which “describes” or defines the configuration of hardware, such as HDL (hardware description language) software, as is used for designing silicon chips, or for configuring universal programmable chips, to carry out desired functions.

The preferred features may be combined as appropriate, as would be apparent to a skilled person, and may be combined with any of the aspects of the invention.

### BRIEF DESCRIPTION OF THE DRAWINGS

Embodiments of the invention will be described, by way of example, with reference to the following drawings, in which:

FIG. 1a is a flow diagram illustrating an example of selecting a data model configuration according to some embodiments of the invention;

FIG. 1b is a schematic diagram illustrating another example of selecting a data model configuration according to some embodiments of the invention;

FIG. 2 is a schematic diagram illustrating another example of optimising a data model configuration iteratively according to some embodiments of the invention;

FIG. 3 is a schematic diagram of an example knowledge graph or subgraph that may be used by the process(es) of FIGS. 1a, 1b and/or 2 and/or a combination thereof;

FIG. 4 is a schematic diagram illustrating an example of selecting a data model configuration for extracting a data model using a knowledge graph and generating predictive models according to some embodiments of the invention;

FIG. 5 is a block diagram illustrating an example of data model configurations with respective scoring;

FIG. 6 is a block diagram of a computing device suitable for implementing some embodiments of the invention.

Common reference numerals are used throughout the figures to indicate similar features.

### DETAILED DESCRIPTION

Embodiments of the present invention are described below by way of example only. These examples represent the best mode of putting the invention into practice that are currently known to the Applicant although they are not the only ways in which this could be achieved. The description sets forth the functions of the example and the sequence of steps for constructing and operating the example. However, the same or equivalent functions and sequences may be accomplished by different examples.

The inventors propose a data model configuration process for identifying and/or selecting the most appropriate data model configuration for creating and/or extracting corresponding data models from a knowledge graph for use in training one or more predictive machine learning (ML) models and/or applying or inputting the data model(s) to train the predictive ML models and the like. In particular, the data model configuration process receives data representative of a plurality of selected data model configuration(s) to create a corresponding plurality of data models from a knowledge graph representing a large data set or corpus associated with, without limitation, for example the biomedical, biological and/or biochemical domains. For simplicity and by way of example only, the knowledge graph may comprise at least a plurality of nodes representing biological entities associated with biomedical, biological and/or biochemical domains, in which each of the nodes are connected by edges to at least one other node, the edges representing relationships between the biological entities. The nodes and/or edges may further include other data and/or attributes that provide further information associated with the nodes, and/or edges and/or relationships therebetween. Each data model of the plurality of data models is used as input for training the same predictive ML model to produce a corresponding plurality of separate trained predictive models. Each of the separate trained predictive models is assessed using benchmarking and/or any other appropriate assessment tool for scoring each separate predictive model. The scoring of a separate trained predictive model is used as a representation of the suitableness of the corresponding data model configuration used to create/extract the data model used to train the separate trained predictive model. Thus, a set of scores for a set of data model configurations is produced that enables a user to select the most appropriate data model configuration for use in extracting a data model from the knowledge graph for training and/or application of said data model to one or more predictive model(s).

This process may be iterated using further data model configuration(s) to identify those data model configuration(s) that result in the best, robust or most suitable data model for use in training or applying to one or more corresponding the same or similar predictive model(s) for solving the same or similar objective problems and the like.

ML technique(s), predictive model algorithms and/or structures may be used to generate a trained predictive model such as, without limitation, for example one or more trained predictive models or classifiers based on input data referred to as training data of known entities and/or entity types and/or relationships therebetween derived from large scale datasets (e.g. a corpus or set of text/documents or unstructured data). With correctly annotated training datasets in the chem(o)informatics and/or bioinformatics fields, ML techniques can be used to generate further trained predictive models, classifiers, and/or analytical models for use in downstream processes such as, by way of example but not limited to, drug discovery, identification, and optimization and other related biomedical products, treatment, analysis and/or modelling in the informatics, chem(o)informatics and/or bioinformatics fields. The term predictive model is used herein to refer to any type of trained model, algorithm or classifier that is generated using a training data set and one or more ML techniques/algorithms and the like.

Specifically, the correctly annotated or labelled training dataset in the chem(o)informatics and/or bioinformatics fields may be retrieved or obtained from various databases, which may be represented as knowledge graphs and the like. These databases/knowledge graphs include but are not limited to the Comparative Toxicogenomics Database (ctdbase.org) and DisGeNET(disgenet.org). Directly and/or indirectly from these databases may be a list of (disease, gene) pairs, or alternatively as a set of triples of the form (disease, confidence score, gene), or a set of quads of the form (disease, relationship type, confidence score, gene). A portion of the data obtained from these databases may be used as a training data set, e.g. by splitting the relationships randomly into two groups, one used for training, and the other one used for the benchmark. Further retrieved data could comprise disease-disease relationships coming from, e.g. an ontology such as Mondo ebb.ac.uk/ols/ontologies/mondo) or the Human Phenotype Ontology (hpo.jax.org). These data would similarly be represented as (disease, disease) pairs, triples of the form (disease, confidence score, disease), or quads of the form (disease, relationship type, confidence score, disease). In this manner, training data sets from, without limitation, for example a knowledge graph may be generated for use with the methods, apparatus and/or system(s) for specifying, testing, evaluating, and selecting data models/data model configurations based on the predictive performance (or other properties) of corresponding predictive ML models trained using training data sets specified by each of the data models/data configurations.

Examples of ML technique(s)/model structure(s) or algorithm(s) for generating a trained predictive model that may be used by the invention as described herein may include or be based on, by way of example only but is not limited to, one or more of: any ML technique or algorithm/method that can be used to generate a trained predictive model based on a labelled and/or unlabelled training datasets; one or more supervised ML techniques; semi-supervised ML techniques; unsupervised ML techniques; linear and/or non-linear ML techniques; ML techniques associated with classification; ML techniques associated with regression and the like and/or combinations thereof. Some examples of ML techniques/model structures may include or be based on, by way of example only but is not limited to, one or more of active learning, multitask learning, transfer learning, neural message parsing, one-shot learning, dimensionality reduction, decision tree learning, association rule learning, similarity learning, data mining algorithms/methods, artificial neural networks (NNs), autoencoder/decoder structures, deep NNs, deep learning, deep learning ANNs, inductive logic programming, support vector machines (SVMs), sparse dictionary learning, clustering, Bayesian networks, reinforcement learning, representation learning, similarity and metric learning, sparse dictionary learning, genetic algorithms, rule-based machine learning, learning classifier systems, and/or one or more combinations thereof and the like. Additionally or alternatively, ML techniques or algorithms/methods that are applicable may be made specifically configured or designed for receiving a graph data structure(s) as input. More specifically, the ML techniques may receive input data such as, without limitation, for example, input data based on a knowledge graph or knowledge graph data structure or data representative of a knowledge graph either directly or indirectly and/or as the application demands.

A knowledge graph and/or entity-entity graph may comprise or represent a graph structure including a plurality of entity nodes in which each entity node is connected to one or more entity nodes of the plurality of entity nodes by one or more corresponding relationship edges, in which each relationship edge includes data representative of a relationship between a pair of entities. The term knowledge graph, entity-entity graph, entity-entity knowledge graph, graph, or graph dataset may be used interchangeably throughout this disclosure.

An entity may comprise or represent any portion of information or a fact that has a relationship with another portion of information or another fact. For example, in the biological, chem(o)informatics or bioinformatics space(s) an entity may comprise or represent a biological entity such as, by way of example only but is not limited to, a disease, gene, protein, compound, chemical, drug, biological pathway, biological process, anatomical region or entity, tissue, cell-line, or cell type, or any other biological or biomedical entity and the like. In another example, entities may comprise a set of patents, literature, citations or a set of clinical trials that are related to a disease or a class of diseases. In another example, in the data informatics fields and the like, an entity may comprise or represent an entity associated with, by way of example but not limited to, news, entertainment, sports, games, family members, social networks and/or groups, emails, transport networks, the Internet, Wikipedia pages, documents in a library, published patents, databases of facts and/or information, and/or any other information or portions of information or facts that may be related to other information or portions of information or facts and the like. Entities and relationships may be extracted from a corpus of information such as, by way of example but is not limited to, a corpus of text, literature, documents, web-pages; a plurality of sources (e.g. PubMed, MEDLINE, Wikipedia); distributed sources such as the Internet and/or web-pages, white papers and the like; a database of facts and/or relationships; and/or expert knowledge base systems and the like; or any other system storing or capable of retrieving portions of information or facts (e.g. entities) that may be related to (e.g. relationships) other information or portions of information or facts (e.g. other entities) and the like; and/or any other data source and/or content from which entities, entity types and relationships of interest may be extracted.

For example, in the biological, chem(o)informatics or bioinformatics space(s), a knowledge graph may be formed from a plurality of entities in which each entity may represent a biological entity from the group of: from the disease, gene, protein, compound, chemical, drug, biological pathway, biological process, anatomical region or entity, tissue, cell-line, or cell type, clinical trials, any other biological or biomedical entity and the like. Each of the plurality of entities may have a relationship with another one or more entities of the plurality of entities or itself. Thus, a knowledge graph or an entity-entity graph may be formed with entity nodes, including data representative of the entities and relationship edges connecting entities, including data representative of the relations/relationships between the entities. The knowledge graph may include a mixture of different entities with data representative of different relationships therebetween, and/or may include a homogenous set of entities with relationships therebetween.

Although details of the present disclosure may be described, by way of example only but are not limited to, with respect to biomedical, biochemical, biological, chem(o)informatics or bioinformatics entities, knowledge or entity-entity graphs and the like it is to be appreciated by the skilled person that the details of the present disclosure are applicable as the application demands to any other type of entity, information, data informatics fields and the like. For simplicity, the following describes a knowledge graph based on, for example, but is not limited to, gene and disease entities.

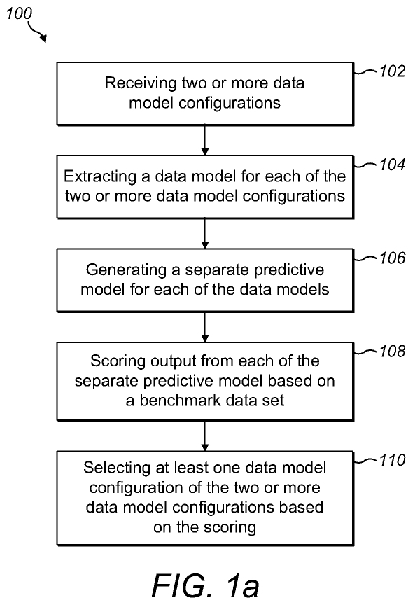

FIG. 1a is a flow diagram illustrating an example data model configuration selection process 100 according to some embodiments of the invention. The data model configuration selection process 100 outputs a set of data model configurations and corresponding scores highlighting the suitability of each data model generated/created. A data model configuration may be selected based on the scoring for use in training one or more predictive model(s) and/or for applying to one or more trained predictive model(s) and the like. The steps of the data model configuration process 100 are as follows:

In step 102, receiving two or more data model configurations in relation to extracting a data model from large-scale data set or corpus represented by a knowledge graph. This may involve receiving multiple data model configurations, and each data model configuration is different from any of the other data model configurations. The data model configuration may comprise or represent data representative of, without limitation, for example one or more constraints or relationships for use in extracting the data model from the knowledge graph.

In step 104, creating and/or extracting a data model for each of the two or more data model configurations from the knowledge graph. Each extracted data model may comprise or represent data representative of a subset of the knowledge graph that is extracted based on the corresponding data model configurations. For example, each extracted data model may comprise or represent a set of training data based on a subset of the knowledge graph extracted from the knowledge graph using a data extraction mechanism configured according to the corresponding data model configuration. The training data may be used for training one or more predictive model(s). Alternatively or additionally, each extracted data model may comprise or represent a set of input data based on a subset of the knowledge graph extracted from the knowledge graph using a data extraction mechanism configured according to the corresponding data model configuration. The input data may be configured for input to one or more trained predictive models.

In step 106, generating a separate predictive model for each of the extracted data models. Each predictive model may be generated by using the corresponding extracted data model to train said predictive model. Although each separate predictive model is trained based on the same ML technique, predictive model algorithm and/or structure, each separate predictive model has been trained using a different data model. A plurality of trained predictive models is generated, with each predictive model having been trained using a different data model. Thus, a plurality of trained predictive models may be generated in which each trained predictive model corresponds to a particular one of the two or more data model configurations. That is, there is a one-to-one mapping between each trained predictive model and a data model configuration of the two or more data model configurations.

In step 108, scoring the output of each separate predictive model based on a benchmark data set. Once trained, each separate predictive model may be assessed as to how well it performs on the specified prediction task[s] using one or more benchmark tests and/or criteria. The scoring of each separate predictive model may be used to represent a scoring for each corresponding data model configuration of the two or more data model configurations. Thus, each data model configuration may be provided with a score based on the scoring of the corresponding trained predictive model based on the data model derived for that data model configuration.

For example, the benchmark data set may include a labelled data set of known inferences or known relationships and/or facts and the like. The benchmark data set is applied to said each of the trained separate predictive model(s), each of which output one or more predictions such as, without limitation, for example at least one relationship inference in relation to the input benchmark data set. The set of predictions output from each trained separate predictive model based on the benchmark data set is compared and scored against the benchmark data set. The scoring for each trained separate predictive model may be expressed as an overall score value or metric derived from, without limitation, for example one or more score value(s) or metric(s), a range of score value(s) or metric(s), a combination of score value(s) or metric(s), and/or a weighted combination of score value(s) and/or score metric(s) and the like. One or more score value(s) or metric(s) may be derived from, without limitation, for example data representative of the accuracy of the set of predictions, the number of false positives and/or false negatives, and/or any other scoring metric and the like used for measuring the output prediction performance, accuracy, robustness, and/or how well the trained predictive model outputs predictions that are accurate in relation to the benchmark data set.

The scoring for each predictive model may include data representative of an overall score and/or a range of one or more score values or metrics. The scoring of each corresponding separate predictive model may be mapped to, assigned and/or attributed to the corresponding data model configuration that was used to generate/extract the data model used for training said corresponding separate predictive model. Thus, each data model configuration of the two or more data model configurations is mapped to or assigned the scoring of the corresponding predictive model.

In step 110, at least one data model configuration of the two or more data model configurations may be selected based on the scoring of each corresponding separate predictive model. The performance of each of the trained separate predictive models is reflected in the scoring; thus the suitability of each of the two or more data model configurations is determined based on the scoring of the corresponding trained separate predictive model. Selecting the data model configuration of the two or more data model configurations based on the scoring may further include, without limitation, for example, selecting the data model configuration based on the output score assigned to a predictive model in relation to the one or more predictions generated by the predictive model in comparison to the benchmark data set. Alternatively or additionally, based on the output scores or scoring of each corresponding separate predictive model, predictive models themselves may be selected as such, to the extent that at least one predictive model and corresponding data model configuration of the two or more data model configurations may be selected based on the output scores.

Thus, selecting a data model configuration from the two or more data model configurations may include selecting a data model configuration based on, without limitation, the highest overall score assigned to each data model configuration and/or one or more scores or metrics associated with each data model configuration and the like. As an option, a corresponding one or more separate predictive models may be selected that correspond to, without limitation, for example the highest overall score assigned to each separate predictive model and/or corresponding selected one or more data model configurations that are considered based on the highest overall score.

In step 104, extracting each data model may include extracting data representative of a subset of the knowledge graph using a data extraction mechanism such as a set of filters associated with or configured according to said each data model configuration, and obtaining a set of training data output based on each extracted subset. The set of training data may be configured to be suitable for input to the separate predictive model for training said separate predictive model.

The data extraction mechanism may include the set of filters used to extract the subset. The set of filters may be configured based on one or more properties or attributes of the knowledge graph and may be used to filter the knowledge graph and extract the subset of the knowledge graph based on the properties or attributes. The properties of the knowledge graph may be, without limitation, for example associated with a proportion of relationships between nodes of the knowledge graph. The proportion of relationship between nodes of the knowledge graph may be limited by one or more constraints set in relation to the properties of the knowledge graph. For example, one or more constraints are associated with types of relationship in the knowledge graph.

In step 106, generating the separate predictive models, for each of the data models, may include, without limitation, for example: tuning each separate predictive model to process each corresponding data model; more specifically or optionally, tuning user-specified parameters of each separate predictive model to optimally handle each corresponding data model; training said each separate predictive model based on applying each corresponding data model to the input of the separate predictive model; and outputting a trained predictive model for use in the scoring step 108. Each separate predictive model may adapt to the amount of training data and type of training data of each of the data models.

As an option, a user or automated process may be configured to tune (or re-tune) each separate predictive model to be optimised for each data model configuration. For example, in the case of much larger data models being used, additional parameters (e.g. model hyperparameters and the like) may be added to the predictive model algorithm and/or structure in which once the data model is created/extracted from the knowledge graph, an iterative training process of using the training data from the particular data model to train each of the separate predictive model(s) based on the corresponding tuned/re-tuned the predictive model algorithm and/or structure.

In step 108, scoring the output from each of the separate trained predictive models based on a benchmark data set may include, without limitation, for example: generating one or more predictions from each separate predictive model based on the benchmark dataset and/or data model that generated the trained predictive model; and comparing the generated one or more predictions with a benchmark set of predictions to obtain a score (e.g. benchmark score) for each of the separate predictive models. In an example, the one or more predictions for each separate trained predictive model may be generated using at least a portion of the benchmark data set applied or input to said each trained separate predictive model, where the predictions that are output are scored based on the expected output from the corresponding portions of the benchmark data set. A benchmark, for example, may comprise a set of known links between genes and diseases, and an evaluation may involve querying the trained predictive model using that set of diseases in the benchmark to get a ranked list of genes for each query disease, and then evaluating the results relative to the known genes in the benchmark.

Step 110 may be further modified to include outputting the at least one selected data model configuration based on the output scores assessed in relation to one or more criteria. This may include outputting each of the data model configuration(s) and the associated scoring assigned to each data model configuration. Additionally or alternatively, outputting and selecting at least one of the data model configuration(s) may further include displaying the data model configuration(s) in relation to the scorings assigned to each data model configuration. The scoring for each data model configuration may be used to assess each of the one or more data model configurations based on one or more criteria, without limitation, for example, data representative of at least one from the group of: a score, or more specifically an accuracy comprising a number of false positives, number of false negatives, a ranking, and any other metric, for example, a performance metric for each of the at least one data model configurations. The score may also be a quality assessment score. For example, the data model configurations for selection may be output as one or more experimental group(s) based on the output scores/scoring, which are assessed in relation to the one or more criteria. The experimental groups may be displayed against the scoring for each data model configuration enabling comparison of the overall scoring and/or one or more scores/metrics making up the overall scoring for selection of the most suitable data model configuration as the application demands or selecting at least one predictive model and corresponding data model configuration.

As an option, the data model configuration process 100 may be iterated in which a user or automated process may be configured to re-tune each separate predictive model to be optimised for each selected data model configuration output from step 110. Thus, steps 102, 104, 106, 108 and 110 may be repeated, in which the selected data model configurations from the previous iteration of step 110 are used along with a re-tuning of the predictive models with re-tuning of the parameters and/or additional parameters (e.g. model hyperparameters and the like) being added to the predictive model structure for each data model of a selected data model configuration, where once the data model is created/extracted from the knowledge graph, each separate predictive model that is re-tuned is retrained using an iterative training process based on using the training data from the particular data model for that separate predictive model to train each of the separate predictive model(s). In this manner, each data model configuration along with the hyperparameters etc., of each separate predictive model may be assessed and this information output along with the scoring relating to the efficacy of each data model/data model configuration.

Thus, in an iterative version of data configuration process 100, the steps of receiving 102, extracting 104, generating 106, scoring 108 and selecting 110 may be performed for each iteration of an iterative data configuration process. The iterative data configuration process may include at least two or more iterations, where for a j-th iteration, j>1, of the at least two or more iterations, the received two or more data configurations may include those selected data model configurations output from the previous (j−1)-th iteration and/or include other data configurations that are to be tested/assessed. The selected data model configuration(s) of the final iteration may be considered an optimised set of data model configuration(s) each of which produces a predictive model with a highest overall score or a plurality of performance statistics that outperform the plurality of performance statistics of other predictive models/data model configurations/data models of any of the previously received data model configuration(s) from any of the at least two or more iterations. Alternatively or additionally, a separate predictive model may be generated and selected from iterating a set of predictive models for each data model configuration/model such that output of each separate predictive model may be scored based on a benchmark data set until a set of ranked predictive models from the set of predictive models and corresponding data models is obtained. From this, the final data model configuration(s) and/or a set of ranked predictive models may be output and/or displayed to a user or output as data representative of a table for selection by a user and/or automated selection process. Alternatively or additionally, an automated selection process may be configured to select the most appropriate data model configuration and/or separate predictive model from the selected data model configuration(s) and/or output data model configuration(s) of step 110 based on various performance criteria and/or statistics that may be required for a future predictive model, a future predictive model within a drug discovery workflow process and the like, and/or as the application demands.

Further to the steps of receiving 102, extracting 104, generating 106, scoring 108 and selecting 110 may be, for example, performed for each iteration of an iterative data configuration process for generating each predictive model. This may include performing the steps of receiving a set of predictive models, generating each predictive model based on one or more data model configurations that have already been selected, scoring each generated predictive model, and selecting one or more predictive models based on the scoring for each iteration of an iterative process comprising at least two or more iterations, wherein for a k-th iteration of the at least two or more iterations, the received set of predictive models comprise the selected predictive models from the previous (k−1)-th iteration; wherein the selected set of predictive models of the final iteration are the predictive models and corresponding data model configurations that produce one or more predictive model(s) ranked with the highest score of the previously received predictive model(s) from any of the at least two or more iterations.

FIG. 1b is a flow diagram illustrating another example data model configuration selection process 120 according to the invention based on data model configuration selection process 100 described with reference to FIG. 1a. The data model configuration selection process 120 is based on the data model configuration selection process 100 described with reference to FIG. 1a. For simplicity, reference numerals from FIG. 1a of similar or the same features, steps and/or components may be reused where applicable. In this example, a knowledge graph 122 containing a large dataset, without limitation, for example a large dataset pertaining to biochemistry, is to be examined to infer new relationships and the like. Although any labelled training data set derived from known relationships and the like of the knowledge graph may be used to train a predictive model, the resulting predictive model is unlikely to provide robust and/or accurate inferences and the like when operating on unknown data from the knowledge graph for, without limitation, for example inferring new relationships and the like. The data model configuration process 120 is a process for searching for the best or suitable data model configuration that may be used to extract a data model for training a predictive model that results in robust and accurate inferences and/or predictions and the like. Using the knowledge graph 122, the steps of data model configuration process 120 are as follows:

In step 124, in order to analyse the knowledge graph 122 data sets, in step 124, a user or an automatic data configuration generation process may select two or more data model configurations for use in generating corresponding data models derived from the knowledge graph 122. Each data model configuration may, without limitation, by way of example be based on selecting data representative of one or more data from the group of: one or more parameters of the knowledge graph, one or more attributes of the knowledge graph, a set of relationships between nodes of the knowledge graph, a set of edges between nodes of the knowledge graph, a filter or limit on the confidence score of certain edges that describe the relationships between nodes, a selection of only certain types of edges of the knowledge graph, or any number of other methods enabling the full knowledge graph to be pruned, sampled, or down-sized to obtain a subset knowledge graph of the available entities and/or relationships. Each data model configuration of the two or more data model configuration(s) are different, which result in different subsets of the knowledge graph. As another example in a biomedical context, a first data model configuration may include only disease—gene edges, whereas a second data model configuration may include the selection of disease—gene edges and disease—disease edges, and a third data model configuration may include only those disease-gene edges with a certain confidence threshold attribute and the like. These first, second and third data model configurations may be used to extract a data model from the knowledge graph. Other examples, in terms of relationship attributes that could be used to generate subset edges of the knowledge graph may include, without limitation, for example the number of evidence sources, the strength of the relationship (e.g. the correlation between two gene expression values), and/or the directionality of the relationship and the like. The data model configuration comprises or represents data representative of how the knowledge graph may be pruned, sampled, and/or down-sized to obtain a subset of the knowledge graph that is useful for training a predictive model and/or useful for applying to a trained predictive model for inferring new relationships as the like.

In step 126, the two or more data model configurations are used, by a data extraction mechanism, to extract two or more data models from the knowledge graph. Each of the two or more data models define a subset of the knowledge graph 122. In step 128, each extracted data model 128a-128n may include a set of training data from the knowledge graph for use in generating a corresponding predictive model. In step 130, the two or more extracted data models 128a-128n may be fed or applied to two or more corresponding predictive models 130a-130n, each of which have been configured based on the extracted data model 128a-128n to infer new relationships between entities in a knowledge graph. For example, in step 130, a predictive model structure, algorithm, or approach is defined and/or selected for inferring new relationships and each of the data model(s) 128a-128n that is extracted from the knowledge graph 122 is separately applied to the predictive model structure to generate a corresponding plurality of separate predictive model(s) 130a-130n. The separate predictive model(s) 130a-130n may be trained or otherwise instantiated. Alternatively, the knowledge graph 122 can be inputted to the predictive model structure to generate the separate predictive model(s) 130a-130n. That is, a first data model 128a is applied to the selected predictive model structure to generate a first trained predictive model 130a, a second data model 128b is applied to the same selected predictive model structure to generate a second trained predictive model 130b, and so on, until the n-th data model 128n is applied to the same selected predictive model structure to generate an n-th trained predictive model 130n. An example of a predictive model (in a biomedical context) may be a predictive model for predicting new genetic drug targets based on the relationships between diseases and genes. Thus, a predictive model structure (e.g. neural network, tensor factorization algorithm, or the like) is defined for use in generating a predictive model for predicting new genetic drug targets based on a labelled training data set. Each of the data model(s) 128a-128n are separately applied to the same predictive model structure for training a corresponding predictive model 130a-130n. Thus, in step 132, each of the two or more data model(s) 128a-128n may be applied to each of the trained predictive models 130a-130n to output a corresponding plurality of sets of predictions 132a-132n of new relationships. For example, in a biomedical context, these may be predictions of inferred disease—gene edges between different entities.

In step 134, each of the trained predictive models 130a-130n are assessed based on a benchmark dataset 136 of known relationships or a benchmark dataset with high confidence (e.g. systematically-extracted or for a genome-wide experimental dataset where individual datapoints are not manually checked, etc.) relationships in which the predictive output of each trained predictive model 130a-130n is able to be scored. Thus, each predictive model 130a-130n is assessed and scored. Given each predictive model 130a-130n was trained or configured using a different corresponding data model 128a-128n, the scoring of each of the predictive models 130a-130n is indicative of the corresponding data model configuration and data model. In order to score each predictive model 130-a-130n, the benchmark dataset 136 may be applied to each of the trained predictive models 130a-130n and the accuracy of the output predictions scored. For example, the benchmark dataset 136 may be processed into a form suitable for each predictive model 130a-130n, which may be based on the corresponding data model 128a-128n. Thus, the benchmark dataset may be applied to each of the trained predictive models 130a-130n, which each output a corresponding plurality of sets of predictive outputs 132a-132n. The corresponding sets of prediction outputs 132a-132n from each trained predictive model 130a-130n are compared with the benchmark data set 136 in order to evaluate the accuracy of each predictive model 130a-130n. For example, the accuracy or scoring of a predictive model 130a may be represented by a score based on the similarity of the output predictions of the predictive model 130a in relation to the benchmark dataset 136. This accuracy evaluation for each of the predictive model(s) 130a-130n may include, without limitation, for example, data representative of one or more score(s), metric(s), a rank, or any other metric for scoring predictive models and the like. For example, the score(s) or metric(s) may be based on one or more predictive model performance statistic(s) including, without limitation, for example, data representative of accuracy, false-positives and/or false-negatives, the precision of each predictive model, or the recall of each predictive model and/or any other score or metric for evaluating the performance of a predictive model. The scoring for each of the predictive models 130a-130n may be output as, without limitation, for example an overall score, an overall score based on a weighted combination of one or more score(s), metric(s) and/or performance statistic(s), and/or a data structure including an overall score and one or more individual score(s), metric(s), performance statistic(s) associated with assessing the performance of each predictive model. For example, the scoring data structure may be based on, without limitation, for example a table of scores in which: each row of the table represents a predictive model 130a-130n; and each column represents an overall score and/or one or more individual scores, metrics, or performance statistics associated with the predictive model 130a-130n.

As an example, in a biomedical context, it may be found that: a first data model configuration defined using only disease—gene edges corresponds to extracting a data model 128a that generates a predictive model 130a that identifies new disease-gene relationships with 80% accuracy; a second data model configuration defined using disease—gene edges and disease—disease edges corresponds to extracting a data model 128b that generates a predictive model 130b that identifies new disease-gene relationships with 85% accuracy. This then enables an automated system and/or user/subject matter expert to select the most accurate or suitable data model configuration (e.g. the second data model configuration) for application to further knowledge graphs for generating data models, that may be used for training or applying to one or more further prediction models for outputting further predictions, and/or to be used in other contexts. Alternatively or additionally, the most accurate or suitable data model configurations may be used as a proxy for selecting the corresponding optimal predictive model (e.g. model with the fastest convergence and/or model with the highest score(s) on validation dataset, etc.) that resulted from the use of the various data models generated using the corresponding data model configuration(s) or with 1-to-1 correspondence with them in upon evaluation process. In either of these cases, the data model configuration process 120 can be used to determine which of a plurality of different data model configurations may be the best or most suitable data model configuration that will or is most likely to generate the most robust predictions from a prediction model, and/or most likely to be used for generating a robust trained prediction model. This avoids a user and/or automated process from wasting time and computing resources and/or guessing which data model configuration is the most effective data model configuration that will result in the most suitable or robust prediction model for any given prediction problem/objective prediction problem and the like.

For example, in step 124, a user may select the two or more desired data model configurations they believe might be effective using a graphical user interface. This may be performed for each designed data model configuration via a GUI process of dragging-and-dropping, or otherwise selecting data representative of desired parameters, attributes, relationships and/or configurations of the knowledge graph from a list of potential relationships, nodes, edges, attributes, filters and/or limits that may be used to generate a suitable subset of the knowledge graph. Given there may be many different combinations of selections for defining a data model configuration for a given prediction model/problem, there may be multiple different data model configurations in which a user and/or automated process cannot be certain is the most effective of use with the same prediction model or for inferring the same type of relationship or prediction problem etc. Thus, with a user-friendly GUI, the data model configuration process 120 reduces manual effort, cognitive load, and room for error in setting and defining two or more desired data model configurations and for properly setting up quick “experiments” (e.g. steps 126-134) for assessing each of the two or more desired data model configurations for, without limitation, for example identifying the most effective data model configuration and/or sanity checking one or more data model configurations and the like. Each experiment may be related to, without limitation, for example one of each of the two or more data model configurations. Additionally or alternatively, an experiment may be related to one full iteration of the data model configuration process 120, which outputs the results of the experiment as a listing of data model configurations and corresponding scoring.

In another example of using data model configuration process 120 according to the invention, a user may define a set of desired relationships to be considered in an experiment in which a set of data model configurations listing possible combinations of these relationships may be produced for the user to select from. During the production of the set of data model configurations, the user may select the number of relationships from an initial list that may be included in each combination. For example, a user may select N (e.g. ten) relationships related to drug discovery, and then specify that N−1 (e.g. nine) of these relationships should be tested at a time. From this, N (e.g. ten) data model configurations could be produced and evaluated using steps 128 to 138, in which each data model configuration excludes one type of relationship. Thus, a user could assess the impact that each relationship has on drug discovery.

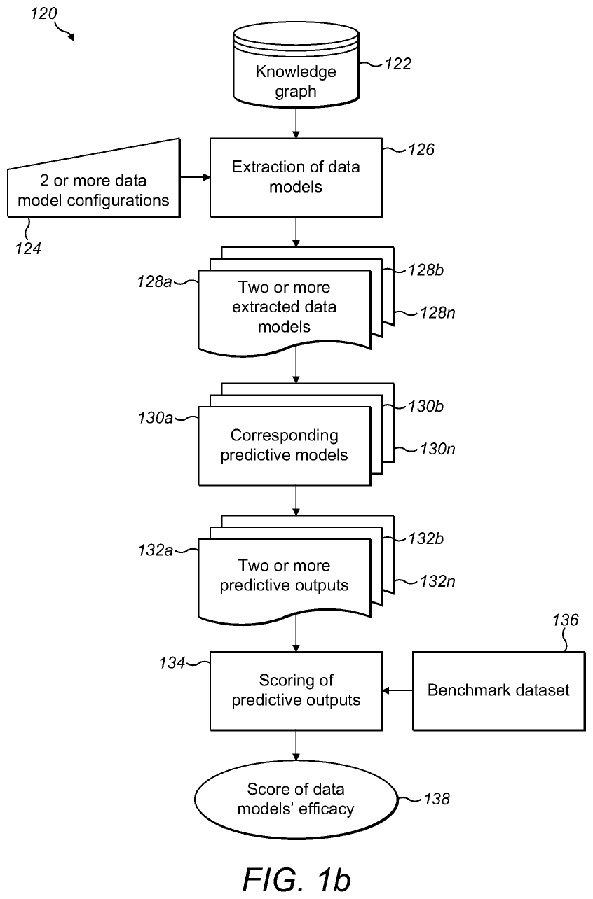

FIG. 2 is another flow diagram illustrating an example iterative data model configuration process 200 according to the invention. The iterative data model configuration process 200 builds upon the data model configuration process(es) 100, 120 as described with reference to FIGS. 1a and 1b. In particular, the data model configuration process 120 of FIG. 1b is further enhanced by iterating over multiple “experiments” to identify the most suitable or best (or optimum) data model configurations 202 for generating data models for applying to the corresponding predictive model(s). For example, the iterative data model configuration process 200 may be configured to iterate the steps of 124, 126, 128, 130, 132, 134, where each iteration uses a different set of data model configurations that generate corresponding data models from the knowledge graph 201 for use with the corresponding separate predictive model(s) for determining a score of each data model configuration and/or data model efficacy that has been iterated. Thus, in response to receiving two or more data model configurations, a set of data model configurations may be optimised until an optimum data model configuration set is obtained, from which a user or automated process may select the most suitable data model configuration to be used with the intended predictive model as the application demands. Optionally, an alternative data predictive model may be obtained for each of the extracted data models from the set of predictive models. This may be derived either directly or indirectly from the set of predictive models for use with any predictive model and the like as the application demands. In this example, in general, the iterative data model configuration process 200 may include the following steps of:

In step 202, a set of data model configurations may be received or sent by a user or automated process. Each data model configuration of the set of data model configurations relates to a different data model that will be used with one or more predictive models and assessed. In step 203, each data model configuration is used to extract a corresponding data model from the knowledge graph 201. It is noted that the knowledge graph 201 may be continually or periodically updated, hence the same data model configuration may produce a different data model with additional updated data than from a previous iteration depending on how often the knowledge graph 201 is updated. The knowledge graph 201 could be updated, for example, based on the continually updated body of research that is published in the field(s) associated with the knowledge graph 201 based on research performed worldwide and/or published in the scientific literature, white papers, articles, journals libraries and the like. For example, the knowledge graph 201 may be associated with biological entities such as, without limitation, for example gene, disease, protein or any other biological entities and relationships thereto. Thus, the knowledge graph 201 may be derived from any text corpus or collection of text sources that are selected from or updated either directly or indirectly based on, without limitation, for example daily updates of and/or publications of biological/biomedical research and/or any other associated research from, without limitation, for example PubMed, conference/journal articles, biological literature, bioinformatics and/or chem(o)informatics literature, relevant databases and/or patents/patent applications and the like. Alternatively or additionally, the knowledge graph 201 may be further updated based on changes to the methodology, for example, of extracting relations from the corpus. The entity nodes and relationship edges of the knowledge graph 201 may be updated in a continual or periodic/aperiodic fashion and so may grow and/or change as the scientific research associated with the knowledge graph 201 grows and/or changes and the like.

In step 204, at least the steps of 128, 130 and 132 of the data configuration process 120 (or at least the steps of 106 and 108 of process 100) may be performed for using each of the extracted data models to generate corresponding predictive models and/or being applied to corresponding predictive models. Where the corresponding predictive models are configured to output inferred relationships and/or predictions associated with the knowledge graph 201 based on the data model used. As an option, in step 204, each of the separate predictive model(s) may be re-tuned and/or tuned using one or more configurable settings of the predictive model. These configuration settings may depend on, without limitation, for example the amount and type of training data being fed in, hyperparameters of the predictive model structure that are being used and the like. Examples of configuration settings may include but are not limited to, for example the number of dimensions used to embed entities and relationships for each data model (when more data is available, a larger embedding space is required to capture all the nuances of the data), as well as parameters/hyperparameters that affect the number of layers, cost functions, step sizes, regularisation, parameters restricting overfitting, i.e. when more data is present, there is less of a requirement to regularize and restrict the model from overfitting.

Thus, in each iteration of the iterative data model configuration process 200, a user or automated process may be configured, in addition to setting or selecting the data model configurations 202, to also provide data representative of tuning parameters and/or re-tune the predictive model(s) used in step 204 to optimise the system for the selected data model configurations 202. For example, in the case of much larger data models being used, additional parameters (e.g. model hyperparameters and the like) may be added to the predictive model. Furthermore, it may be that for a set of data model configurations that are being compared, the predictive model is tuned to optimally process each data model. This would happen after the data model is created/extracted from the knowledge graph 201, and consists of, for example in step 204, an iterative training process of using the training data from the particular extracted data model (from step 203) to train various versions of the predictive model in step 204.

Steps 205, 206 and 207 may be based on steps 134, 136 and 138 of the data model configuration process 120. For example, in step 205 each of the configured or trained predictive model(s) in step 204 are assessed based on a benchmark dataset 206 of known (or otherwise manually-checked) relationships in which the predictive output of each trained predictive model is scored. This scoring for each predictive model is reflective of the suitability or scoring for each corresponding data mode/and/or data model configuration. In step 207, the efficacy of each of the data model configurations and/or data model(s) in the set of data model configurations provided in step 202 are scored based on the scoring of each of the corresponding predictive model(s). In step 208, one or more data model configurations from: a) the set of data model configurations that are provided in step 202; and/or b) that have been provided in previous iterations of the iterative data model configuration process 200 may be selected based on the scoring of the corresponding data model. The selected set of data model configurations/data models may be considered the optimum set of data model configurations for use with one or more predictive models and/or for training future predictive models and the like. The selected set of data model configurations/data models may further include data representative of the tuning parameters and/or re-tuning parameters used in relation to the predictive models when assessed.

Step 208 may feed back into step 202 of the iterative data model configuration process 200, in which further set of data model configurations may be selected/set for assessment in a further iteration in relation to one or more data models and corresponding predicted models and the like. The set of data model configurations in step 202 may be augmented by one or more of the selected data model configurations from step 208, where the corresponding predictive model might be re-tuned and/or retrained. Furthermore, the assessment of the selected and/or optimum set of data model configurations may need to be reassessed due to updates to the knowledge graph 201 and/or by re-tuning the predictive model, and/or the user changing the predictive model to another type of predictive model that may be applicable with the selected and/or optimum set of data model configurations.

Thus, the iterative data configuration process 200 may be further modified in steps 205, 207 and/or 208, in which the resultant comparison of different configurations may be output as an experimental group, with visualisations that illustrate, for each data model configuration/data model, the overall scoring and/or the different/various scores, metrics or performance statistics of the corresponding predictive model(s) to enable comparisons of each data model configuration/data model and the like. For example, one of the visualisations may be a graph showing the accuracy metrics associated with each data model configuration. Such visualisations may be used for selecting one or more data model configurations for further assessment, analysis and/or use. For example, a user may be running many experiments (e.g. an experiment may correspond to an iteration of steps 203, 204 and 205 of process 200 in which a set of data model configurations is assessed), and within each experiment (e.g. iterative run of steps 203, 204 and 205) there is a set of two or more data model configurations that will produce two or more data models for the user or an automated process to assess and determine the most suitable data model/data model configuration(s) that may be used with the particular predictive model and possible future predictive models that the user may be implementing. Therefore it is important to be able to group each experiment appropriately and to make the appropriate and/or proper statistical comparisons between the data model configuration(s) under assessment for each particular/specific predictive model.

In a biomedical example, one visualisation may illustrate the difference between a first data model configuration that considers only disease—gene edges of the knowledge graph 201 and a second data model configuration that considers disease—gene edges and disease—disease edges of the knowledge graph. The differences may be visualised in a table of data model configurations/data models with corresponding performance statistics in relation to the corresponding predictive model that uses that data model configuration/data model.

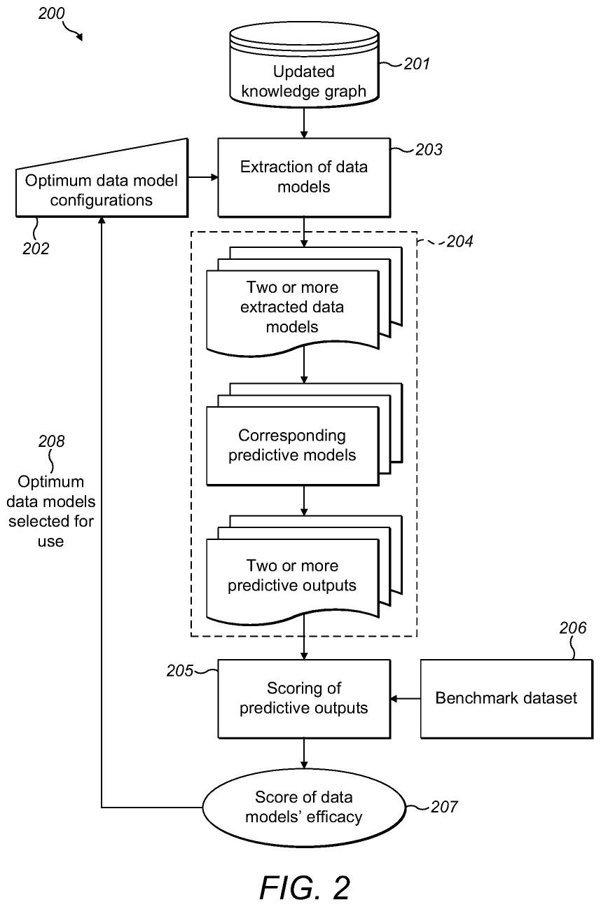

FIG. 3 is a schematic diagram illustrating a portion of an example knowledge graph 300 for use with the data model configuration process and/or system according to the invention. The knowledge graph 300 includes a plurality of nodes 301, 303 and 304 (also referred to herein as entity nodes) connected with one or more other nodes to a plurality of edges 302, 305 and 306. The plurality of nodes 301, 303, 304 represent entities (e.g. Entity 1, Entity 2, Entity 3), which may be, without limitation, for example biological entities and the like, and the plurality of edges 302, 305 and 306 represent relationships that connect the nodes 301, 303, 304. Each of the edges 302, 305 and 306 may represent a relationship that associates a node of the plurality of nodes 301, 303, 304 with another of the plurality of nodes 301, 303, 304. Note, it is also possible to have knowledge graphs in which a node is self-connected by an edge, i.e. an edge that loops back to connect with the same node. Each of the edges 302, 305, 306 may include further attributes associated with the relationship such as, without limitation, for example directionality, labelling, the confidence score of the relationship, and any other useful information associated with the relationship and the like etc.

In this example, a first entity node 301 representing a first entity, e.g. Entity 1, is linked via a first edge 302 to a second entity node 303 representing a second entity, e.g. Entity 2, where the first edge 302 is labelled, without limitation, for example with data representing the form of the relationship that exists between the first and second entities, e.g. Entity 1 and Entity 2, of the first and second entity nodes 301 and 303, respectively. For example, in the biomedical domain, the first entity (e.g. Entity 1) of the first entity node 301 may be a gene and the second entity (e.g. Entity 2) of the second entity node 303 may be a disease. Thus, the edge 302 between the first and second entity nodes 301 and 303 may be configured, in this example, to represent a gene-disease relationship, which, without limitation, for example may be tantamount to “causes” if the gene (Entity 1) of the first entity node 301 is responsible for the presence of the disease (Entity 2) of the second entity node 303.

Expanding on this example, if the third entity node 304 represents a third entity (e.g. Entity 3) that may also be a disease in which shared a disease—disease relationship exists over edge 305 with the second entity (e.g. Entity 2) of the second entity node 303. Given this, a trained predictive model may be configured to examine the knowledge graph and infer new gene-disease relationships and so, may on receiving data representative of a portion or subset of the knowledge graph representing nodes 301, 303 and 304 connected with edges 302 and 305, infer or predict a new gene-disease relationship represented by dashed edge 306 between the first entity (e.g. Entity 1) of the first entity node 301 and the third entity (e.g. Entity 3). Thus, new edge 306 may be inferred by the trained predictive model being trained and/or examining a data model configured to include data representative of the knowledge graph 300 represented by nodes 301, 303 and 304 and edges 302 and 302 as depicted in FIG. 3. However, these new inferences may not always prove to be correct; thus, as detailed above, a predictive model may be run based on using different data model configurations to generate different data models representing knowledge graph 300 in which the resultant sets of predictions, when compared to a benchmark dataset are used to evaluate each different data model configurations' accuracy or the suitability of each different data model configuration based on how the predictive model performs using said each different data model generated from the corresponding data model configuration.

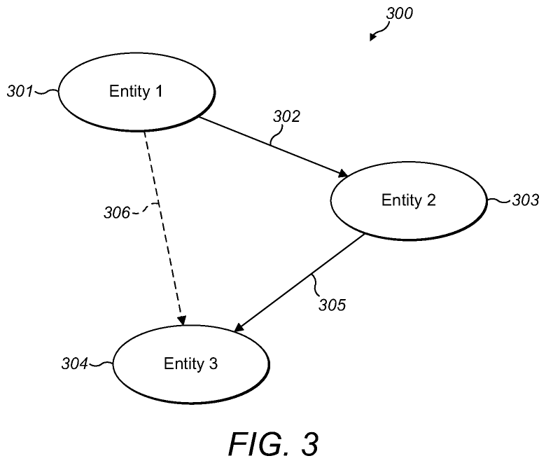

FIG. 4 is a schematic diagram illustrating a data model configuration system 400 according to the invention. The data model configuration system 400 may use the data model configuration process(es) 100, 120 and/or 200 as described with reference to FIGS. 1a to 2. The data model configuration system 400 includes a knowledge graph 401, a data model configuration component 402, a data model extraction component 403, a prediction model component 404, and an assessment and selection component 405. The data model configuration system 400 may be configured to perform a single pass for assessing and selecting a set of data model configurations/data models as herein described and/or may be configured to perform an iterative feedback loop for assessing and selecting a set of data model configurations. The data model configuration component 402 is configured to receive two or more data model configurations from a user, automated process and/or from a selection of two or more data model configurations from a previous iteration of the data model configuration system 400 output from assessment and selection component 405. The data model configuration component 402 feeds the set of data model configurations to a data model extraction component 403, which also receives a knowledge graph 401. The data model extraction component 403 operates on the knowledge graph 401 and the set of data model configurations to extract a corresponding set of data model(s). Each data model includes data representative of a subset knowledge graph of the knowledge graph 401 extracted based on the corresponding data model configuration from the set of data model configurations. Thus, a plurality of data model(s) is extracted by the data model extraction component 403 in which each data model is different from another of the plurality of data models. Each of the set of extracted data model(s) includes a subset of the knowledge graph 401 that is derived from the corresponding data model configuration. Each subset of the knowledge graph 401 may be divided into one or more training data sets, testing data sets, and/or validation data sets and the like.

Each of the extracted data model(s) is provided by the data model extraction component 403 to the prediction model component 404. The prediction model component 404 is configured to generate a plurality of predictive models based on each of the extracted data model(s). As described previously, this may involve generating a plurality of predictive models, one predictive model for each data model of the set of data models. This may be achieved by, without limitation, for example using a common ML technique, predictive model algorithm and/or structure to generate, for each data model of the set of data models, a trained predictive model using the training data set of said each data model. Thus, a plurality of trained predictive models is generated, each trained based on the training data set of the corresponding extracted data model. As described, each extracted data model may include data representative of a training data set, a validation data set and/or an input data set for use with the trained predictive model, which has been trained and/or updated based on the training data set. Although each of the plurality of predictive models is based on the same or a common ML technique/predictive model algorithm or structure, they are different in the sense that they have been trained and/or updated using a different data model and/or configured to use a different data model from the set of extracted data models. Each of the predictive models are configured to receive as input the extracted data model and output, without limitation, corresponding predictions, classifications, and/or infer relationships and the like associated with the knowledge graph 401.

In the case predictions, classifications, and/or infer relationships, the training data set may be from a structured database such as the Comparative Toxicogenomics Database (ctdbase.org) or DisGeNET (disgenet.org), and could be represented either as a list of (disease, gene) pairs, or alternatively as a set of triples of the form (disease, confidence score, gene), or quads of the form (disease, relationship type, confidence score, gene). This represented list, set or quad of data can be used for training in this example, and any examples herein described, e.g. by splitting the relationships randomly into two groups, one used for training, and the other one used for the benchmark or validation. Additional training data could comprise disease-disease relationships coming from, e.g. an ontology such as Mondo (ebb.ac.uk/ols/ontologies/mondo) or the Human Phenotype Ontology (hpo.jax.org). These would similarly be represented as (disease, disease) pairs, triples of the form (disease, confidence score, disease), or quads of the form (disease, relationship type, confidence score, disease).

The assessment and scoring component 405 receives each of the predictive models generated by the predictive model component 404 for assessing using benchmark data sets. The benchmark data sets may be derived from the knowledge graph 401. Each predictive model of the plurality of predictive models is assessed and scored by the assessment and scoring component 405. The scoring for each trained predictive model is indicative of the performance of that predictive model based on the benchmark data set. This scoring may include scores, metrics and/or performance statistics for assessing the accuracy of the predictions and/or inferences output from the predictive model based on the corresponding input benchmark data set. The scoring for each predictive model is used to assess the efficacy of the corresponding data model configuration and/or data model used in relation to said each predictive model. Thus, scoring results may include data representative of a table with each row representing a data model configuration and corresponding data model/predictive model us and each column representing one or more scores or an overall scoring of the predictive model performance based on the benchmark data set. Thus, a user and/or an automated process may assess the scoring results and select one or more data model configurations/data models according to a set of performance criteria such as, without limitation, for example data representative of the highest overall scoring, highest accuracy score, least number of false positives and/or false negatives, and/or a selection of scores, metrics and/or performance statistics associated with the data model configuration and corresponding predictive model.

The scoring results may be stored and/or appended to previous scoring results to enable a user and/or automated process to assess all data model configurations that have been tested with corresponding predictive models and the like. This enables further selection of the most suitable or appropriate data model configuration in relation to a particular prediction model or a particular type of prediction model algorithms/structures used to generate a prediction model and the like.

Additionally or alternatively, a selection of one or more of the data model configurations that have been assessed by the assessment and scoring component 405 may further provided to the data model configuration component 402, where these data model configurations may be added to a further set of data model configurations in which the corresponding predictive models and/or predictive model algorithms/techniques may be further tuned, re-tuned in an effort or attempt to further improve the performance of the resulting predictive models when used with the corresponding data model extracted based on the selected one or more data model configurations. The data model configuration system 400 performs further processing on the further set of data model configurations and knowledge graph 401 using the data model extraction component 403, the predictive model component 404, and assessment and scoring component 405 in relation to the further set of data model configurations.

Alternatively or additionally, as an option the selection of one or more data model configurations based on the efficacy of the data model/predictive model may be selected and used for implementation and/or development of future predictive models and/or algorithms and the like. For example these may be provided to a workflow process for drug discovery in which one or more optimal data model configurations are selected for use with one or more predictive models in a drug discovery system/workflow process and the like.

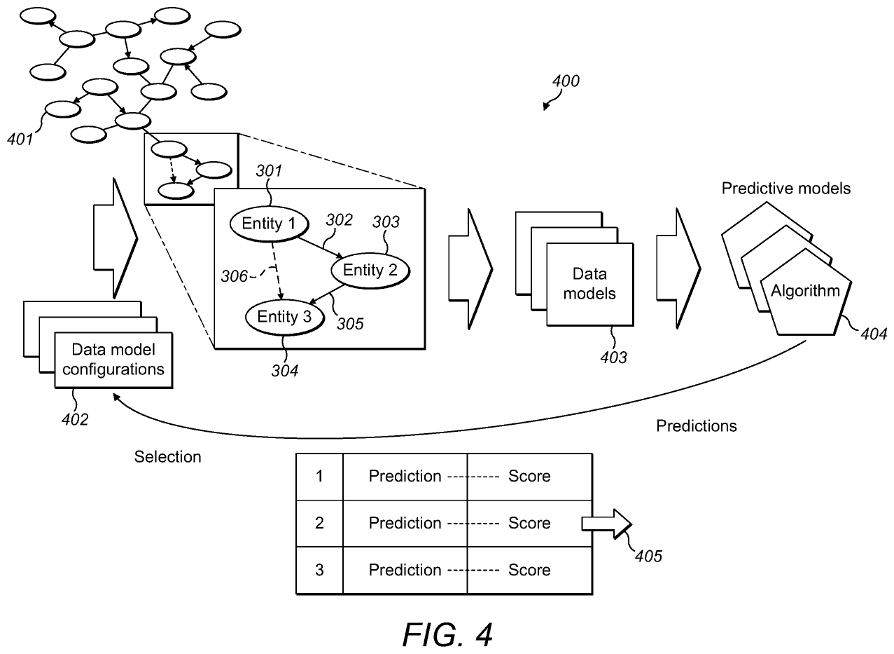

FIG. 5 is a schematic illustration of an example scoring results data structure 500 output from the data model configuration system 400 of FIG. 4 and/or output from the data model configuration process(es) 100, 120 and/or 200 of FIGS. 1a to 2. In this example, the scoring results data structure 500 is illustrated as a table data structure with each row representing a data model configuration/data models of a plurality of data model configurations(s)/model(s) 501-504, and each column representing a scoring associated with the predictive model generated or configured by the data model corresponding to each data model configurations 501-504.

As described previously, the data model configuration comprises or represents data representative of how the knowledge graph may be pruned, sampled, and/or down-sized to obtain a subset of the knowledge graph that is useful for training a predictive model and/or useful for applying to a trained predictive model for inferring new relationships and the like. In this example, there are four data model configurations 501-504 which are used for predicting disease-gene links or relationships. Each of the four data models would be evaluated individually to predict new or unseen disease-gene relationships. A portion of the disease— gene relationships are reserved for the training or as training dataset. Accordingly, the first data model configuration 501 may include every disease-gene relationship (or edges), whereas a second data 502 model configuration may include only the selection of disease— gene edges and gene-disease edges with a high confidence score (e.g. confidence score >0.5), a third data model configuration 503 may include every disease-gene edges and only gene-gene edges with a certain confidence threshold attribute and the like, and the fourth data model configuration 504 may include disease-gene edges (confidence >0.5) and gene-gene edges. These first, second, third, and fourth data model configurations may be used to extract a data model from the knowledge graph. Examples in terms of relationship attributes that could be used to generate a subset edges of the knowledge graph may include, without limitation, the number of evidence sources, the strength of the relationship (e.g. the correlation between two gene expression values), and/or the directionality of the relationship and the like. The data model configuration comprises or represents data representative of how the knowledge graph may be pruned, sampled, and/or down-sized to obtain a subset of the knowledge graph that is useful for training a predictive model and/or useful for applying to a trained predictive model for inferring new relationships as the like. Accordingly, the four different data models may be extracted from the knowledge graph based on the corresponding data model configuration. Each of the four data models will include a different subset of the knowledge graph based on the definition of the corresponding data model configuration 501-504. Each of the four data models is used with the same or similar predictive algorithm or ML technique to configure a trained predictive model corresponding to said each data model. Thus, four different predictive models based on the same or common predictive model algorithm and/or ML technique is output in which each predictive model is configured or optimised in relation to the corresponding data model. For example, a first predictive model is generated/configured in relation to the first data model configuration 501 based on the first extracted data model; a second predictive model is generated/configured in relation to the second data model configuration 502 based on the second extracted data model; a third predictive model is generated/configured in relation to the third data model configuration 503 based on the third extracted data model; a fourth predictive model is generated/configured in relation to the fourth data model configuration 504 based on the fourth extracted data model; and so on.

The output predictions and/or inferences of each predictive model is assessed and scored using a benchmark data set. The scoring results may be associated with the corresponding data model configuration used to extract the data model used to configure each predictive model. Thus, the performance scorings of each predictive model derived from the benchmark dataset assessment may be tabulated with the data model configuration in the scoring result data structure 500. In this case, the overall scorings for each predictive model are stored in the scoring result data structure 500, which represent the overall accuracy or provide an estimate of the overall performance of the corresponding predictive model and hence the efficacy of the data model configuration. In this example, the first data model configuration 501 is associated with the first predictive model's overall accuracy score of 98%, the second data model configuration 502 is associated with the second predictive model's overall accuracy score of 80%, the third data model configuration 503 is associated with the third predictive model's overall accuracy score of 91%, and the fourth data model configuration 504 is associated with the fourth predictive model's overall accuracy score of 97%. The scoring result data structure 500 may be displayed to the user and/or used by an automated process to select one or more data model configurations of the set of data model configurations 501-504 that are most suitable for use with the predictive model and/or type of predictive model algorithm/technique. As described, one or more of these data model configurations may be fed back and a further set of data model configurations assessed and scored as described by the data model configuration process(es) 100, 120, 200 of FIGS. 1a to 2 and/or data model configuration system, 400 of FIG. 4 and/or as the application demands.

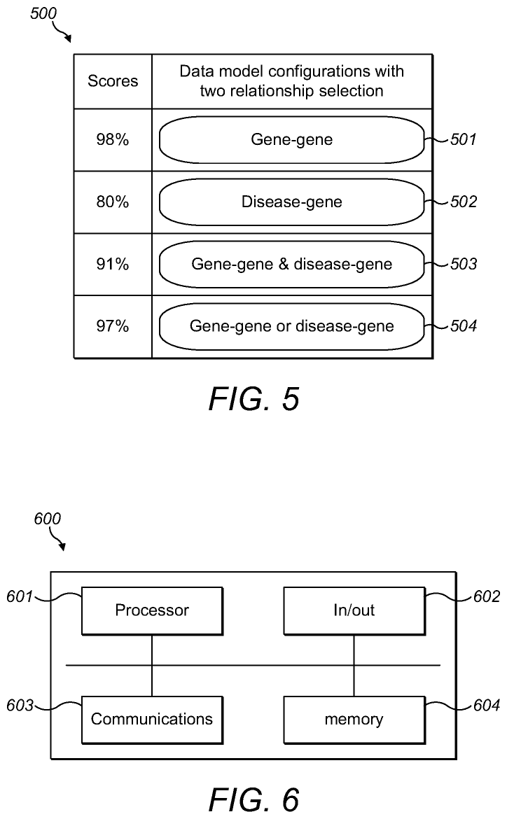

FIG. 6 is a schematic diagram illustrating an example computing apparatus/system 600 that may be used to implement one or more aspects of the data configuration system(s), apparatus, method(s), and/or process(es) combinations thereof, modifications thereof, and/or as described with reference to FIGS. 1a to 5 and/or as described herein. Computing apparatus/system 600 includes one or more processor unit(s) 601, an input/output unit 602, communications unit/interface 603, a memory unit 604 in which the one or more processor unit(s) 601 are connected to the input/output unit 602, communications unit/interface 603, and the memory unit 604. In some embodiments, the computing apparatus/system 600 may be a server, or one or more servers networked together. In some embodiments, the computing apparatus/system 600 may be a computer or supercomputer/processing facility or hardware/software suitable for processing or performing the one or more aspects of the data configuration system(s), apparatus, method(s), and/or process(es) combinations thereof, modifications thereof, and/or as described with reference to FIGS. 1a to 5 and/or as described herein. The communications interface 603 may connect the computing apparatus/system 600, via a communication network, with one or more services, devices, server system(s), cloud-based platforms, systems for implementing subject-matter databases and/or knowledge graphs for implementing the invention as described herein. The memory unit 604 may store one or more program instructions, code or components such as, by way of example only but not limited to, an operating system and/or code/component(s) associated with the data model configuration process(es)/method(s) as described with reference to FIGS. 1a to 5, additional data, applications, application firmware/software and/or further program instructions, code and/or components associated with implementing the functionality and/or one or more function(s) or functionality associated with one or more of the method(s) and/or process(es) of the device, service and/or server(s) hosting the data model configuration process(es)/method(s)/system(s), apparatus, mechanisms and/or system(s)/platforms/architectures for implementing the invention as described herein, combinations thereof, modifications thereof, and/or as described with reference to at least one of FIGS. 1a to 5.

In an aspect associated with FIGS. 1a to 5, a computer-implemented method of selecting a data model configuration for use in training predictive models comprising: receiving two or more data model configurations; extracting a data model for each of the two or more data model configurations from a knowledge graph; generating a separate predictive model for each of the extracted data models; scoring the output of each separate predictive model based on a benchmark data set; and selecting at least one data model configuration of the two or more data model configurations based on the output scores.

In another aspect, a computer-implemented method for training a separate predictive model for each of two or more data model configurations comprising: extracting a set of training data for each of the two or more data model configuration from a knowledge graph; and training the separate predictive model using the set of training data.

In yet another aspect, a computer-implemented method for training a predictive model comprising: selecting a data model configuration from the at least one data model configurations output by any computer-implemented method as optionally described below; extracting a set of training data from a knowledge graph based on the selected data model configuration; and training the predictive model using the extracted set of training data.

In yet another aspect, a ML model or classifier obtained from using training data extracted from a knowledge graph based on a selected data model configuration output from any of the computer-implemented methods that are optionally described below.

In yet another aspect, a computer-readable medium comprising computer-readable code or instructions stored thereon, which when executed on a processor, causes the processor to implement the computer-implemented method as optionally described below.

In yet another aspect, an apparatus comprising a processor, a memory and a communication interface, the processor connected to the memory and communication interface, wherein the apparatus is adapted or configured to implement the computer-implemented method as optionally described below.

In yet another aspect, an apparatus for selecting a data model configuration, the apparatus comprising: an input component configured to receive two or more data model configurations; a processing component configured to extract a data model for each of the two or more data model configurations from a knowledge graph; a prediction component configured to generate a separate predictive model for each of the data models; a scoring component configured to score output from each of the separate predictive model based on a benchmark data set; and a selection component configured to select the data model configuration of the two or more data model configurations based on the scoring. Optionally, the apparatus may be adapted or configured to implement the computer-implemented method as described below. Optionally, the apparatus further comprises a display component configured to visualise scores for comparing each of the two or more data model configurations.

Optionally, selecting at least one predictive model and corresponding data model configuration of the two or more data model configurations based on the output scores.

Optionally, each extracted data model comprises a set of training data based on a subset of the knowledge graph extracted from the knowledge graph using a data extraction mechanism configured according to the corresponding data model configuration.

Optionally, each of the two or more data model configurations comprise data representative of one or more constraints or relationships for use in extracting the data model from the knowledge graph.

Optionally, extracting a data model for each of the two or more data model configurations further comprising: extracting data representative of a subset of the knowledge graph using a set of filters associated with each of the two or more data model configurations; and obtaining a set of training data output for each extracted subset.

Optionally, the set of filters corresponds to properties associated with the knowledge graph.

Optionally, the properties of the knowledge graph is associated with a proportion of relationships between nodes of the knowledge graph.

Optionally, the proportion of relationships between nodes of the knowledge graph are limited by one or more constraints set in relation to the properties of the knowledge graph.

Optionally, the one or more constraints are associated with types of relationship in the knowledge graph.

Optionally, generating the separate predictive model for each of the data models further comprising: tuning each separate predictive model to process each corresponding data model; training said each separate predictive model based on applying each corresponding data model to the input of the separate predictive model; and outputting a trained predictive model for use in scoring.

Optionally, each separate predictive model adapts to the amount of training data and type of training data of each of the data models.

Optionally, scoring output from each of the separate predictive model based on a benchmark data set further comprising: generating one or more predictions from each separate predictive model; and comparing the generated one or more predictions with a benchmark set of predictions to obtain a score for each of the separate predictive model.

Optionally, the one or more predictions are generated using at least a portion of the benchmark data set.

Optionally, selecting the data model configuration of the two or more data model configurations based on the scoring further comprising: selecting the data model configuration based on the score in relation to the one or more predictions generated in comparison to the benchmark set of predictions.

Optionally, the one or more predictions comprise at least one relationship inference amongst the data models extracted.

Optionally, the knowledge graph comprises nodes representing biological entities associated with biomedical or biochemical domains.

Optionally, selecting at least one data model configuration of the two or more data model configurations based on the output scores further comprises: outputting the at least one selected data model configurations based on the output scores assessed in relation to one or more criteria.

Optionally, the data model configuration is output as one or more experimental groups based on the output scores assessed in relation to the one or more criteria.

Optionally, displaying the data model configuration in relation to the one or more experimental group.

Optionally, the one or more criteria comprise at least one from the group of: a score, a ranking, and a metric for each of the at least one data model configuration.

Optionally, iterating the steps of selecting for the data model configuration using the separate predictive models in response to receiving two or more data model configurations to be optimised until an optimum data model configuration set is obtained.

Optionally, performing the steps of receiving, extracting, generating, scoring and selecting for each iteration of an iterative process comprising at least two or more iterations, wherein for a j-th iteration of the at least two or more iterations, the received two or more data configurations comprise the selected data model configuration output from the previous (j−1)-th iteration; wherein the selected data model configuration of the final iteration is the data model configuration that produces a predictive model with highest score of the previously received data model configuration from any of the at least two or more iterations.

Optionally, iterating selecting from a set of predictive models and generating a separate predictive model for each of the extracted data models from the set of predictive models, and scoring the output of each separate predictive model based on a benchmark data set until a set of ranked predictive models from the set of predictive models and corresponding data models is obtained.

Optionally, performing the steps of receiving a set of predictive models, generating each predictive model, scoring each generated predictive model, and selecting one or more predictive models based on the scoring for each iteration of an iterative process comprising at least two or more iterations, wherein for a k-th iteration of the at least two or more iterations, the received set of predictive models comprise the selected predictive models from the previous (k−1)-th iteration; wherein the selected set of predictive models of the final iteration are the predictive models and corresponding data model configurations that produces one or more predictive model(s) ranked with highest score of the previously received predictive model(s) from any of the at least two or more iterations.

Optionally, the knowledge graph is updated, when iterating or during the iteration, in relation to the biomedical or biochemical domains.

In the embodiments, examples, of the invention as described above such as data model configuration process(es), method(s), system(s) and/or apparatus may be implemented on and/or comprise one or more cloud platforms, one or more server(s) or computing system(s) or device(s). A server may comprise a single server or network of servers, the cloud platform may include a plurality of servers or network of servers. In some examples the functionality of the server and/or cloud platform may be provided by a network of servers distributed across a geographical area, such as a worldwide distributed network of servers, and a user may be connected to an appropriate one of the network of servers based upon a user location and the like.

The above description discusses embodiments of the invention with reference to a single user for clarity. It will be understood that in practice the system may be shared by a plurality of users, and possibly by a very large number of users simultaneously.

The embodiments described above may be configured to be semi-automatic and/or are configured to be fully automatic. In some examples a user or operator of the data model configuration system(s)/process(es)/method(s) may manually instruct some steps of the process(es)/method(es) to be carried out.

The described embodiments of the invention the data model configuration system, process(es), method(s) and/or apparatus and the like according to the invention and/or as herein described may be implemented as any form of a computing and/or electronic device. Such a device may comprise one or more processors which may be microprocessors, controllers or any other suitable type of processors for processing computer executable instructions to control the operation of the device in order to gather and record routing information. In some examples, for example where a system on a chip architecture is used, the processors may include one or more fixed function blocks (also referred to as accelerators) which implement a part of the process/method in hardware (rather than software or firmware). Platform software comprising an operating system or any other suitable platform software may be provided at the computing-based device to enable application software to be executed on the device.

Various functions described herein can be implemented in hardware, software, or any combination thereof. If implemented in software, the functions can be stored on or transmitted over as one or more instructions or code on a computer-readable medium or a non-transitory medium. Computer-readable media may include, for example, computer-readable storage media. Computer-readable storage media may include volatile or non-volatile, removable or non-removable media implemented in any method or technology for storage of information such as computer-readable instructions, data structures, program modules or other data. A computer-readable storage media can be any available storage media that may be accessed by a computer. By way of example, and not limitation, such computer-readable storage media may comprise RAM, ROM, EEPROM, flash memory or other memory devices, CD-ROM or other optical disc storage, magnetic disc storage or other magnetic storage devices, or any other medium that can be used to carry or store desired program code in the form of instructions or data structures and that can be accessed by a computer. Disc and disk, as used herein, include compact disc (CD), laser disc, optical disc, digital versatile disc (DVD), floppy disk, and blu-ray disc (BD). Further, a propagated signal is not included within the scope of computer-readable storage media. Computer-readable media also includes communication media including any medium that facilitates transfer of a computer program from one place to another. A connection or coupling, for instance, can be a communication medium. For example, if the software is transmitted from a website, server, or other remote source using a coaxial cable, fiber optic cable, twisted pair, DSL, or wireless technologies such as infrared, radio, and microwave are included in the definition of communication medium. Combinations of the above should also be included within the scope of computer-readable media.

Alternatively, or in addition, the functionality described herein can be performed, at least in part, by one or more hardware logic components. For example, and without limitation, hardware logic components that can be used may include Field-programmable Gate Arrays (FPGAs), Program-specific Integrated Circuits (ASICs), Program-specific Standard Products (ASSPs), System-on-a-chip systems (SOCs). Complex Programmable Logic Devices (CPLDs), etc.

Although illustrated as a single system, it is to be understood that the computing device may be a distributed system. Thus, for instance, several devices may be in communication by way of a network connection and may collectively perform tasks described as being performed by the computing device.

Although illustrated as a local device it will be appreciated that the computing device may be located remotely and accessed via a network or other communication link (for example using a communication interface).

The term ‘computer’ is used herein to refer to any device with processing capability such that it can execute instructions. Those skilled in the art will realise that such processing capabilities are incorporated into many different devices and therefore the term ‘computer’ includes PCs, servers, IoT devices, mobile telephones, personal digital assistants and many other devices.

Those skilled in the art will realise that storage devices utilised to store program instructions can be distributed across a network. For example, a remote computer may store an example of the process described as software. A local or terminal computer may access the remote computer and download a part or all of the software to run the program. Alternatively, the local computer may download pieces of the software as needed, or execute some software instructions at the local terminal and some at the remote computer (or computer network). Those skilled in the art will also realise that by utilising conventional techniques known to those skilled in the art that all, or a portion of the software instructions may be carried out by a dedicated circuit, such as a DSP, programmable logic array, or the like.

It will be understood that the benefits and advantages described above may relate to one embodiment or may relate to several embodiments. The embodiments are not limited to those that solve any or all of the stated problems or those that have any or all of the stated benefits and advantages. Variants should be considered to be included into the scope of the invention.

Any reference to ‘an’ item refers to one or more of those items. The term ‘comprising’ is used herein to mean including the method steps or elements identified, but that such steps or elements do not comprise an exclusive list and a method or apparatus may contain additional steps or elements.

As used herein, the terms “component” and “system” are intended to encompass computer-readable data storage that is configured with computer-executable instructions that cause certain functionality to be performed when executed by a processor. The computer-executable instructions may include a routine, a function, or the like. It is also to be understood that a component or system may be localized on a single device or distributed across several devices. Further, as used herein, the term “exemplary”, “example” or “embodiment” is intended to mean “serving as an illustration or example of something”. Further, to the extent that the term “includes” is used in either the detailed description or the claims, such term is intended to be inclusive in a manner similar to the term “comprising” as “comprising” is interpreted when employed as a transitional word in a claim.

The figures illustrate exemplary methods. While the methods are shown and described as being a series of acts that are performed in a particular sequence, it is to be understood and appreciated that the methods are not limited by the order of the sequence. For example, some acts can occur in a different order than what is described herein. In addition, an act can occur concurrently with another act. Further, in some instances, not all acts may be required to implement a method described herein.

Moreover, the acts described herein may comprise computer-executable instructions that can be implemented by one or more processors and/or stored on a computer-readable medium or media. The computer-executable instructions can include routines, sub-routines, programs, threads of execution, and/or the like. Still further, results of acts of the methods can be stored in a computer-readable medium, displayed on a display device, and/or the like.

The order of the steps of the methods described herein is exemplary, but the steps may be carried out in any suitable order, or simultaneously where appropriate. Additionally, steps may be added or substituted in, or individual steps may be deleted from any of the methods without departing from the scope of the subject matter described herein. Aspects of any of the examples described above may be combined with aspects of any of the other examples described to form further examples without losing the effect sought.

It will be understood that the above description of a preferred embodiment is given by way of example only and that various modifications may be made by those skilled in the art.

What has been described above includes examples of one or more embodiments. It is, of course, not possible to describe every conceivable modification and alteration of the above devices or methods for purposes of describing the aforementioned aspects, but one of ordinary skill in the art can recognize that many further modifications and permutations of various aspects are possible. Accordingly, the described aspects are intended to embrace all such alterations, modifications, and variations that fall within the scope of the appended claims.

1. A computer-implemented method of selecting a data model configuration for use in training predictive models comprising:
receiving two or more data model configurations;
extracting a data model for each of the two or more data model configurations from a knowledge graph;
generating a separate predictive model for each of the extracted data models;
scoring an output of each separate predictive model based on a benchmark data set; and
selecting at least one data model configuration of the two or more data model configurations based on the output scores.

2. The computer-implemented method as claimed in claim 1, further comprising selecting at least one predictive model and corresponding data model configuration of the two or more data model configurations based on the output scores.

3. The computer-implemented method as claimed in claim 1, wherein each extracted data model comprises a set of training data based on a subset of the knowledge graph extracted from the knowledge graph using a data extraction mechanism configured according to the corresponding data model configuration.

4. The computer-implemented method as claimed in claim 1, wherein each of the two or more data model configurations comprise data representative of one or more constraints or relationships for use in extracting the data model from the knowledge graph.

5. The computer-implemented method as claimed in claim 1, wherein extracting a data model for each of the two or more data model configurations further comprising:
extracting data representative of a subset of the knowledge graph using a set of filters associated with each of the two or more data model configurations; and
obtaining a set of training data output for each extracted subset.

6. The computer-implemented method as claimed in claim 5, wherein the set of filters corresponds to properties associated with the knowledge graph and wherein the properties of the knowledge graph are associated with a proportion of relationships between nodes of the knowledge graph.

7. (canceled)

8. The computer-implemented method as claimed in claim 6, wherein the proportion of relationships between nodes of the knowledge graph are limited by one or more constraints set in relation to the properties of the knowledge graph and wherein the one or more constraints are associated with types of relationship in the knowledge graph.

9. (canceled)

10. The computer-implemented method as claimed in claim 1, wherein generating the separate predictive model for each of the data models further comprises:
tuning each separate predictive model to process each corresponding data model;
training said each separate predictive model based on applying each corresponding data model to an input of the separate predictive model; and
outputting a trained predictive model for use in scoring.

11. The computer-implemented method as claimed in claim 8, wherein each separate predictive model adapts to an amount of training data and type of training data of each of the data models.

12. The computer-implemented method as claimed in claim 1, wherein scoring output from each of the separate predictive model based on a benchmark data set further comprises:
generating one or more predictions from each separate predictive model; and
comparing the generated one or more predictions with a benchmark set of predictions to obtain a score for each of the separate predictive model,
wherein the one or more predictions are generated using at least a portion of the benchmark data set.

13. (canceled)

14. The computer-implemented method as claimed in claim 12, wherein selecting the data model configuration of the two or more data model configurations based on the scoring further comprises:
selecting the data model configuration based on the score in relation to the one or more predictions generated in comparison to the benchmark set of predictions.

15. The computer-implemented method as claimed in claim 12, wherein the one or more predictions comprise at least one relationship inference amongst the data models extracted.

16. The computer-implemented method as claimed in claim 1, wherein the knowledge graph comprises nodes representing biological entities associated with biomedical or biochemical domains.

17. The computer-implemented method as claimed in claim 1, wherein selecting at least one data model configuration of the two or more data model configurations based on the output scores further comprises:
outputting the at least one selected data model configurations based on the output scores assessed in relation to one or more criteria,
wherein the data model configuration is output as one or more experimental groups based on the output scores assessed in relation to the one or more criteria, and further comprising:
displaying the data model configuration in relation to the one or more experimental groups.

18. (canceled)

19. (canceled)

20. (canceled)

21. The computer-implemented method as claimed in claim 1, further comprising:
iterating the steps of selecting for the data model configuration using the separate predictive models in response to receiving two or more data model configurations to be optimised until an optimum data model configuration set is obtained.

22. The computer-implemented method as claimed in claim 1, further comprising:
performing the steps of receiving, extracting, generating, scoring and selecting for each iteration of an iterative process comprising at least two or more iterations, wherein for a j-th iteration of the at least two or more iterations, the received two or more data configurations comprise the selected data model configuration output from the previous (j−1)-th iteration;
wherein the selected data model configuration of the final iteration is the data model configuration that produces a predictive model with highest score of the previously received data model configuration from any of the at least two or more iterations.

23. The computer-implemented method as claimed in claim 21, further comprising:
iterating selecting from a set of predictive models and generating a separate predictive model for each of the extracted data models from the set of predictive models, and scoring the output of each separate predictive model based on a benchmark data set until a set of ranked predictive models from the set of predictive models and corresponding data models is obtained.

24. The computer-implemented method as claimed in claim 1, further comprising:
performing the steps of receiving a set of predictive models, generating each predictive model, scoring each generated predictive model, and selecting one or more predictive models based on the scoring for each iteration of an iterative process comprising at least two or more iterations, wherein for a k-th iteration of the at least two or more iterations, the received set of predictive models comprise the selected predictive models from the previous (k−1)-th iteration;
wherein the selected set of predictive models of the final iteration are the predictive models and corresponding data model configurations that produces one or more predictive model(s) ranked with highest score of the previously received predictive model(s) from any of the at least two or more iterations.

25. The computer-implemented method as claimed in claim 21, wherein the knowledge graph is updated, when iterating or during the iteration, in relation to the biomedical or biochemical domains.

26. (canceled)

27. (canceled)

28. (canceled)

29. (canceled)

30. (canceled)

31. An apparatus for selecting a data model configuration, the apparatus comprising:
an input component configured to receive two or more data model configurations;
a processing component configured to extract a data model for each of the two or more data model configurations from a knowledge graph;
a prediction component configured to generate a separate predictive model for each of the data models; a scoring component configured to score output from each of the separate predictive model based on a benchmark data set; and
a selection component configured to select the data model configuration of the two or more data model configurations based on the scoring.

32. (canceled)

33. (canceled)
